(19) United States
(12) Patent Application Publication
(10) Pub. No.: US 2005/0203458 A1
(43) Pub. Date: Sep. 15, 2005

(54) Safe retractable type syringe

(75) Inventor: Chao-Hua Shih, Taipei (TW)

Correspondence Address: TROXELL LAW OFFICE PLLC;SUITE 1404, 5205 LEESBURG PIKE, FALLS CHURCH, VA, 22041, US

(73) Assignee: Life-Shield Products, Inc.

(21) Appl. No.: 11/128,258

(22) Filed: May 13, 2005

Related U.S. Application Data
(63) Continuation-in-part of application No. 10/418,062, filed on Apr. 18, 2003.

(51) Int. Cl. A61M 5/00

(52) U.S. Cl. 604/110; 604/198

(57) ABSTRACT

A safe retractable type syringe comprising a needle set, a barrel and a plunger, the needle set is inserted into and connected with the inner wall of the barrel from the front end of the barrel, and is pulled back into the barrel to be connected with the barrel after injection. The syringe is characterized in that, the bottom of the needle set has on the periphery thereof at least one first annular flange, the front end of the barrel has on its inner wall a second annular flange and at least two protruding ribs equidistantly away from the second annular flange; this can improve the state of connecting of the needle set with the barrel, the needle set and the barrel thus are tightly engaged with each other, thereby liquid medicine can be sealed, and the operations to insert with a force and connect and pull the needle set into the barrel are both easy; thereby manufacturing of the engaging structure of the needle set and the barrel can be easier.

This is a continuation-in-part application of the applicant's U.S. patent application Ser. No. 10/418,062 filed on Apr. 18, 2003.

### BACKGROUND OF THE INVENTION

1. Field of the invention

The present invention is related to a safe retractable type syringe; and especially to an engaging structure of a barrel and a needle set retractable into the barrel, wherein the needle set is inserted into and connected with the inner wall of the barrel from the front end of the barrel, and is pulled back into the barrel to be connected with the barrel after injection. The needle set and the barrel are suitably connected, production and controlling are easy, the product of such syringe is convenient, and the operation of inserting as well as pulling back the needle set are easy too.

2. Description of the Prior Art

The U.S. Pat. No. 5,899,887,owned by the same patentee as the present invention, discloses a safe retractable type syringe structure having a changeable needle, referring to FIG. 1, the structure generally is provided with a retractable type bottom seat C of a needle set with a first engaging portion C11 therebeneath, the bottom seat C has on the outside of its upper portion a plurality of flanges C12, a second engaging portion B11 connectable with the first engaging portion C11 is provided on an upper end of a plunger B. After injection, the plunger B received in a barrel A is pushed upwardly to its topmost position to make connecting of the first engaging portion C11 with the second engaging member B11, thereby, the retractable type bottom seat C connected on the upper end of the inner wall of the barrel A is pulled back into the barrel A ready for discarding or disposing. Wherein the first engaging portion C11 of the patent is inserted from the rear of the barrel A, and is firmly connected with the inner wall of the barrel A by mutual connecting of the flanges C12 provided on the outside of the bottom seat C with a plurality of flanges A11 on an inner wall of the barrel A, thus a function that liquid medicine is sealed can be attained. However, in practicing the above U.S. patent, the quality of the syringe made is very hard to control, this is because using a die to shape the syringe by injection molding and by using plastic is necessary, and during the process of die releasing after shaping of the syringe, the flanges are subjected to rubbing with the die, so that the flanges are worn off, and thereby the tolerance between the bottom seat of the needle set and the inner wall of the front end of the barrel is destroyed to render the two unable to be tightly connected with each other, thus a problem is induced, namely, the function that liquid medicine is sealed can not be attained.

And the U.S. Pat. No. 5,217,437 also discloses a needle protecting device, wherein an inner surface of a needle shield 40 is also provided with a plurality of detents 50 able to be received within slots 26 in the triangular protrusions 24 to releasably retain the needle shield 40 in its retracted position (lines 8-13, column 6). And thereby, the needle shield 40 can be moved frontward to cover the needle. However the mode of connecting of the needle shield 40 of such syringe with the needle must be specifically designed; and manufacturers must have their cost of producing such needle shields 40 increased.

In making engaging of a needle seat with a barrel of the present invention, the engaging structure of a needle set and the inner wall of the barrel “A” of a safe retractable type syringe of the present invention must be taken into consideration carefully. In the above U.S. Pat. No. 5,899,887, the engaging of the needle seat with the inner wall of the barrel is unable to be applied to the present invention, because by such mode of engaging, sometimes the needle set is hard to be inserted to engage in the barrel from the front end of the latter, sometimes the needle set is overly loose after it is inserted into the front end of the barrel, if the problem of tolerance in manufacturing is not well controlled; for example, when the outer diameter of the needle set and the inner diameter on the front end of the barrel are not suitably controlled, or the sizes and the positions of the plural flanges on the inner wall of the barrel (there are three flanges disclosed in FIG. 11) are not accurately designed; this may induce a problem in getting superiority of products, and operations in inserting and pulling the needle set into the barrel are not smooth. And engaging of the needle set with the inner wall of the barrel of the above U.S. Pat. No. 5,217,437 is unable either to be applied to the present invention to insert the needle set from the front end of the barrel.

An explanation has to be presented: for a conventional non-retractable type syringe, its needle seat is inserted from the front end of its barrel, this is that the conventional non-retractable type syringe needs no consideration on the problem of pulling the needle set back into the barrel, it needs only to have its needle set inserted and tightly connected. While the main point of the present invention is resided in that: the needle set is directly inserted from the front end of the barrel and is retractable, the structure disclosed in the present invention is suitable for production, quality control and manipulation.

### SUMMARY OF THE INVENTION

The motive of the present invention is to improve the engagement of the needle set with the barrel, wherein the needle set is directly inserted from the front end of the barrel, the needle set and the barrel are tightly engaged with each other, thereby liquid medicine can be sealed. The operations to insert with a force and connect the needle set into the barrel and to pull the needle set into the barrel are both easy. The engaging structure of the needle set with the barrel is benefit to manufacturing of the present invention.

The primary object of the present invention is to provide a safe retractable type syringe; wherein an engaging structure of a needle set and a barrel is improved, the needle set thus can be inserted into and connected with the barrel from the front end of the latter, thereby the product of the present invention is easy for manufacturing, operating and use, and in which liquid medicine is sealed.

The secondary object of the present invention is to provide an engaging structure of the needle set and the barrel for a safe retractable type syringe, the structure is applicable to a safe retractable type syringe either with a standard needle set having a changeable needle or with a needle set having an unchangeable needle.

To achieve the above objects, the present invention is comprised of a needle set, a barrel and a plunger. Wherein the front end of the needle set is connected with a needle, while the rear end is provided with a first engaging portion, the bottom of the needle set is provided on the periphery thereof at least with a first annular flange; the barrel is a hollow pipe, its front end is a socket for connecting thereto of the needle set, the socket is provided on the inner wall thereof with a second annular flange and at least with two protruding ribs equidistantly away from the second annular flange. The rear end of the barrel is used for slipping in the plunger of which the front end is provided with a piston, a second engaging portion is provided on the front end of the piston. Thereby, when the needle set is connected into the needle-set socket from above, the first annular flange provided on the periphery of the needle set is located between the second annular flange provided on the inner wall of the needle-set socket and the two protruding ribs, in order to overcome the problem of tolerance in manufacturing to make easier for manufacturing, assembling and use as well as to increase the effect of sealing liquid medicine. When the front end of the plunger is pushed to the topmost position, the first engaging portion and the second engaging portion connect with each other, so that when the plunger is pulled back, the needle set is pulled back into the barrel.

The present invention will be apparent in its features after reading the detailed description of the preferred embodiments thereof in reference to the accompanying drawings.

### BRIEF DESCRIPTION OF THE DRAWINGS

FIG. 1 is an analytic perspective view showing the elements of a conventional syringe;

FIG. 2 is an analytic perspective view showing the first embodiment of the present invention;

FIG. 3 is a sectional view showing assembling of the elements of the first embodiment of the present invention;

FIG. 3a is an exploded perspective view showing the upper end portion of the first embodiment of the present invention;

FIG. 3b is a sectional view taken from a section line AA in FIG. 3a of the first embodiment of the present invention;

FIG. 4 is a sectional view showing assembling of the second embodiment of the present invention;

FIG. 4a is an enlarged sectional view taken from FIG. 4.

FIG. 5 is a sectional view showing assembling of the elements of another embodiment of the present invention;

FIG. 5a is a partial sectional schematic perspective view of the embodiment of FIG. 5.

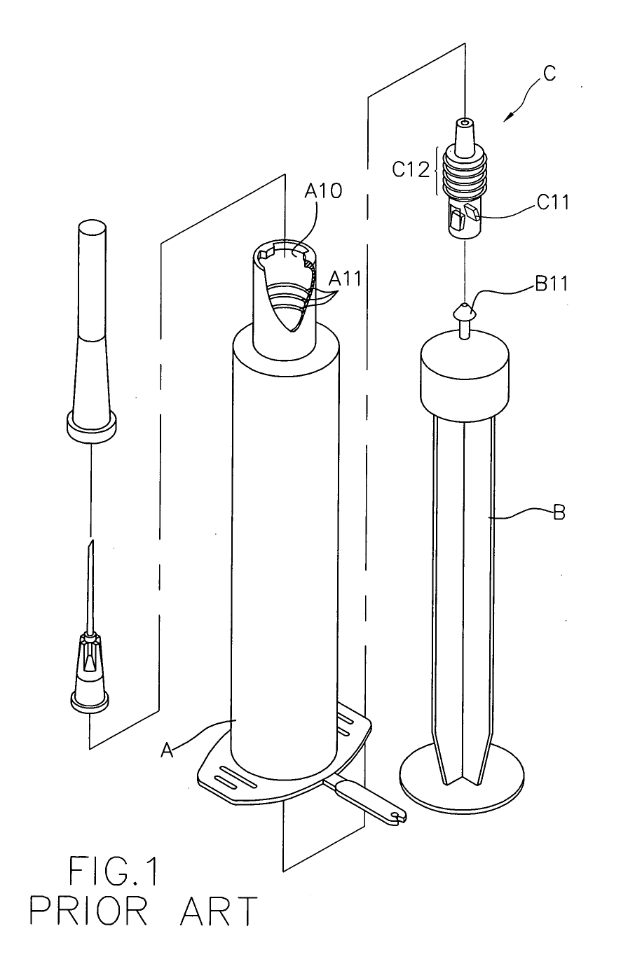

### DETAILED DESCRIPTION OF THE PREFERRED EMBODIMENT

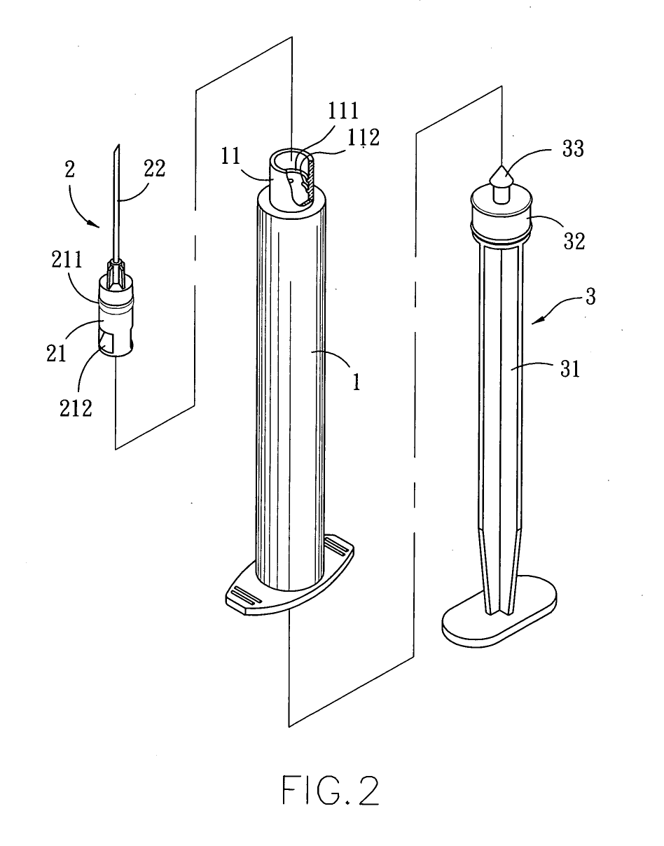

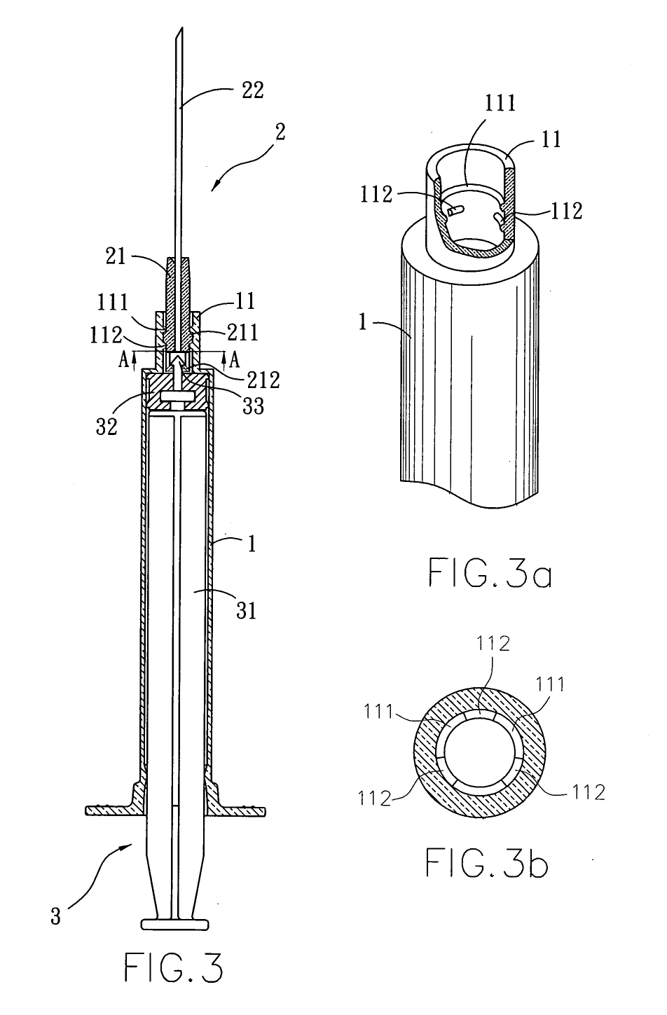

Referring to FIG. 2 showing a first preferred embodiment of the present invention applicable to a safe retractable type syringe with an unchangeable needle, the embodiment comprises a barrel 1, a retractable type needle set 2 and a plunger 3. Wherein the barrel 1 is a hollow pipe, its front end is reduced to form a needle-set socket 11, the needle-set socket 11 is provided on the inner wall thereof with a second annular flanges 111 and with three protruding ribs 112 (in practical, two protruding ribs will be allowable) equidistantly away from the second annular flange 111 such as are shown in FIGS. 3a and 3b; the protruding ribs 112 are all allocated on a circle, in practicing, the protruding ribs 112 are preferably equidistantly spaced.

The retractable type needle set 2 includes a needle seat 21 and a needle 22, the rear end of the needle seat 21 is provided with a first engaging portion 212; the external diameter of the needle seat 21 is coincident with the inner diameter of the needle-set socket 11, so that the needle seat 21 can be connected with the latter, and the way of connection is that the needle seat 21 is inserted into the barrel 1 from the front end of the needle-set socket 11 of the barrel 1; the external wall of the middle section of the needle seat 21 is provided at least with a first annular flange 211 to increase the sealing function in connecting. In this embodiment, the first engaging portion 212 is an engaging portion with a recess, while the recess is formed from more than one detent piece.

The plunger 3 is generally a pusher rod 31, the front end thereof has a piston 32; a second engaging portion 33 is provided on the front end of the piston 32. In this embodiment, the second engaging portion 33 is a protruding hook portion. The plunger 3 is provided for slipping into the barrel 1 to move forward and backward therein.

Referring to FIGS. 3 and 3a, the retractable type needle set 2 is inserted into the needle-set socket 11 of the barrel 1, and the first flange 211 of the needle seat 21 is engaged between the second flange 111 of the needle-set socket 11 and the three protruding ribs 112 equidistantly away from the second annular flange 111, so that the retractable type needle set 2 can be more tightly connected with the needle-set socket 11. When the retractable type safe syringe is used, it shall be sealed during the processes both of medicine drawing out and injection; while after injection, the plunger 3 is pushed to its topmost position, the second engaging portion 33 provided on the front end of the piston 32 can be connected with the first engaging portion 212 of the needle seat 21 and the protruding hook portion of the second engaging portion 33 is engaged with the ends of the detent pieces. At this time, the plunger 3 is pulled back; then the retractable type needle set 2 including the needle 22 can be drawn back into barrel 1.

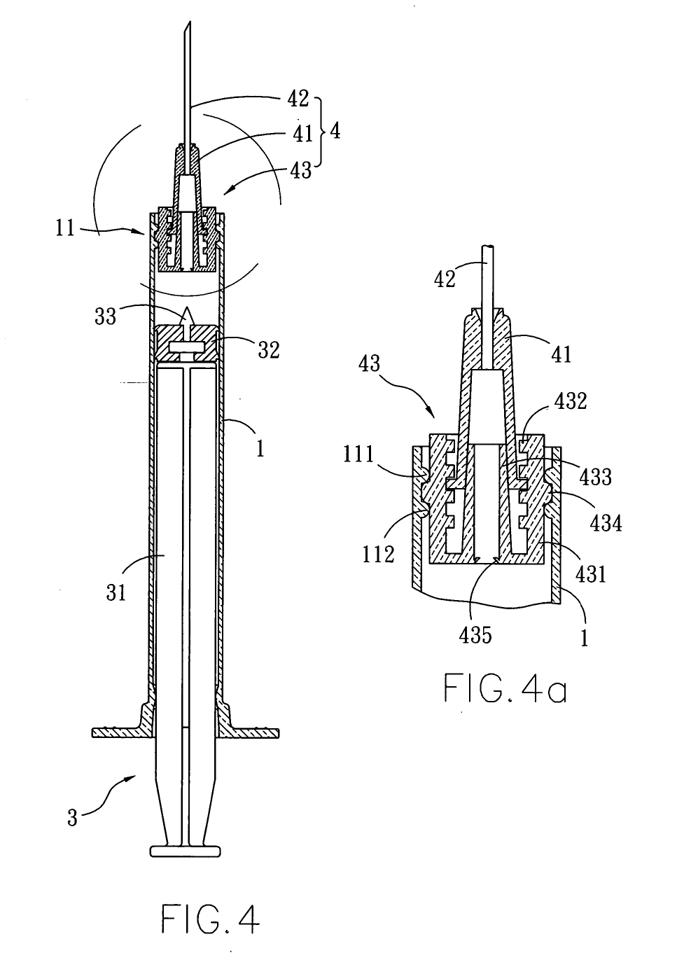

Referring to FIGS. 4 and 4a, the second embodiment of the present invention with a changeable needle is comprised of a barrel 1, a standard needle set 4 and a plunger 3; wherein the barrel 1 is a hollow pipe, its front end has a needle-set socket 11; the needle-set socket 11 is provided on the inner wall thereof with a second flange 111 of the needle-set socket 11 and three protruding ribs 112 (in practical, two protruding ribs will be allowable) equidistantly away from the second annular flange 111. As shown in FIG. 4a, the standard needle set 4 includes a needle seat 41, a needle 42 and a bottom seat 43. The needle 42 is provided on the front end of the needle seat 41 which can be detached from and connected to the bottom seat 43 of the needle set 4; the bottom seat 43 is provided with an annular housing 431 and a hollow inner pipe 433. The inner wall of the annular housing 431 has a thread 432 for screw connecting the needle seat 41, while external wall of the annular housing 431 has at least one annular flange 434. The rear end of the hollow inner pipe 433 has a first engaging portion 435; in this embodiment, the needle seat 41 and the bottom seat 43 are rotating connected as a Lure lock, while in practicing, it can alternatively be engaged directly with the standard needle set 4 as a Lure slip structure.

The standard needle set 4 is inserted into the needle-set socket 11 provided on the front end of the barrel 1. The first flange 434 of the bottom seat 43 is engaged between the second flange 111 of the needle-set socket 11 and the three protruding ribs 112, so that the syringe will be sealed during the processes of medicine drawing out and injection. When the front end of the plunger 3 is pushed to the topmost position after injection, the second engaging portion 33 on the front end of the piston 32 is connect with the first engaging portion 435 of the bottom seat 43 to pull the plunger 3 rearwardly, and the standard needle set 4 can then be pulled into the barrel 1.

The above stated in referring to the drawings are preferred embodiments of the present invention, for example, wherein the first engaging portion 212 of the needle seat 21 on the retractable type needle set 2 and the first engaging portion 435 of the bottom seat 43 of the standard needle set 4 are both provided with recesses; while the second engaging portion 33 on the plunger 3 is provided with a protruding hook portion. However, in practicing, other shapes and structures able to get the function of engagement can alternatively be adopted. By all means, the first engaging portion 212, 435 respectively of the needle seat 21 of the retractable type needle set 2 and the bottom seat 43 can be provided with hook portions, while the second engaging portion 33 on the plunger 3 can be provided with a recess, and the same function of engagement can likely be achieved.

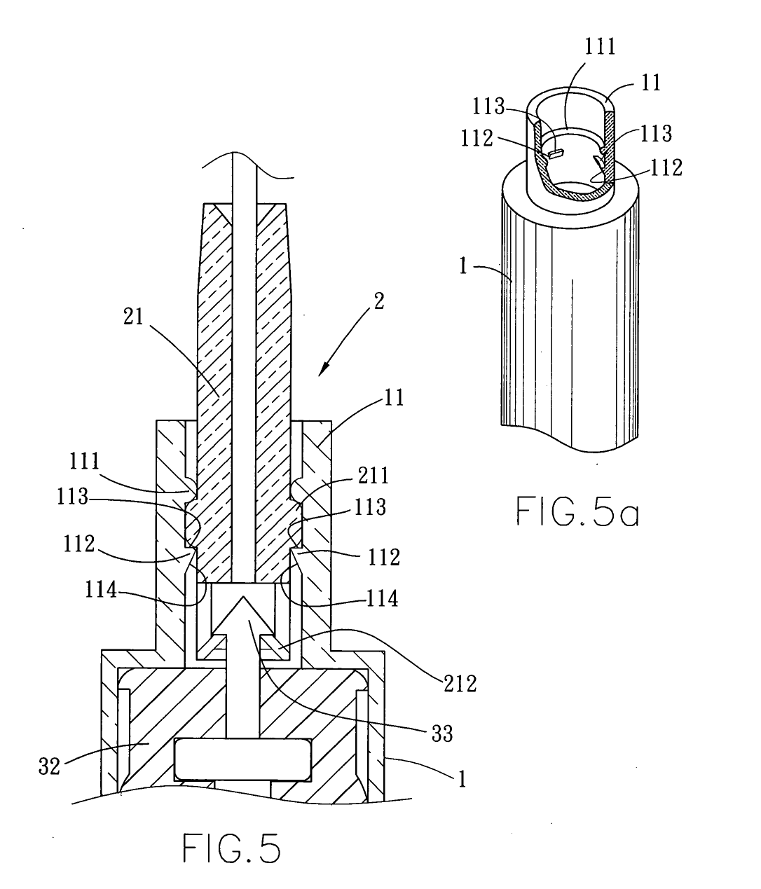

In operation, when the needle of the syringe is inserted into the skin surface of a man, normally an impeding force is induced against injection, the impeding force will make contracting of muscle in different degrees according to the degrees of nervousness of the person being injected, and normally the value of the impeding force is about 0.6 kg, but sometimes the muscle becomes stiff because of nervousness of the person, the value probably reaches 1.0 kg, or even higher. Thereby in practicing, referring to FIGS. 5 and 5a, wherein a barrel 1 has the three protruding ribs 112 on the inner wall of its front end preferably in the shapes of triangles each having a supporting surface 113 and a bevel surface 114, the supporting surface 113 is generally perpendicular to the front end of the barrel 1, the supporting surface 113 cooperates with the bevel surface 114 to abut against and support the first flange 211; and the supporting surface 113 has a length that decides the magnitude of the supporting force for the needle seat 21. Generally, designing of the length renders it able to bear 1.6 kg impeding force. In practicing the present invention, the needle seat 21 is subjected to a force to be inserted from the front end of the barrel 1 into the needle-set socket 11, the first flange 211 of the needle seat 21 passes through the second flange 111 on the inner wall of the needle-set socket 11, and then is abutted by the supporting surfaces 113 of the three protruding ribs 112 to be engaged between the second flange 111 and the three protruding ribs 112. After injection, the second engaging portion 33 provided on the front end of the piston 32 is connected with the first engaging portion 212 of the needle seat 21; then a user exerts a force larger than 1.6 kg to pull the needle seat 21 back into the barrel 1, at this time, the supporting surfaces 113 of the three protruding ribs 112 are worn out by rubbing with the first flange 211 of the needle seat 21 to allow pulling back of the needle seat 21.

Accordingly, the present invention has the following advantages:
1. The safe retractable type syringe of the present invention is provided on the periphery of the bottom of the needle set at least with a first annular flange; the front end of the barrel is provided on the inner wall thereof with a second annular flange and at least with two protruding ribs equidistantly away from the second annular flange. The state of connecting of the improved needle set with the barrel allows the needle set to be inserted from the front end of the barrel, and the needle set and the barrel are tightly engaged with each other, thereby liquid medicine can be sealed.
2. With the safe retractable type syringe of the present invention, the operations to insert with a force and connect the needle set into the barrel and to pull the needle set into the barrel are both easy.
3. With the safe retractable type syringe of the present invention, the engaging structure of the needle set with the barrel can overcome the problem of tolerance in manufacturing to make easier for manufacturing the syringe.

As stated in the above disclosed, the present invention can surely attain its expected objects to provide an improved engaging structure of a retractable type needle set and a barrel, the structure undoubtedly has its practical value of utility.

It will be apparent to those skilled in this art that various modifications or changes made to the elements of the present invention without departing from the spirit of this invention shall fall within the scope of the appended claims.

1. A safe retractable type syringe comprising:
a needle set, a front end of said needle set being connected with a needle, while a rear end of said needle set being connected with a first engaging portion;
a barrel, being a hollow pipe, a front end thereof being a needle-set socket provided for connecting said needle set therein; and
a plunger in the form of a pusher rod, a front end thereof having a piston, said plunger being provided for slipping into said barrel to move forward and backward therein for drawing out or injecting liquid medicine; a second engaging portion being provided on a front end of said piston and connected with said first engaging portion;
when said front end of said plunger being pushed to a topmost position, said first engaging portion and said second engaging portion connecting with each other, so that when said plunger being pulled back, said needle set being pulled back into said barrel;
wherein said needle-set socket is provided on an inner wall thereof with a second annular flange and provided below said second annular flange at least with two protruding ribs equidistantly away from said second annular flange; a bottom of said needle set is provided on a periphery thereof at least with one first annular flange, so that when said needle set is inserted and connected with said needle-set socket, said at least one first flange provided on said periphery of said bottom of said needle set is located between said second annular flange provided on said inner wall of said needle-set socket and said at least two protruding ribs.

2. The safe retractable type syringe as claimed in claim 1, wherein said needle set is a retractable type needle set with an unchangeable needle and comprises said needle and a needle seat, said needle is fixed on a front end of said needle seat, said needle seat is a bottom seat of said needle set, said front end of said barrel is reduced to form said needle-set socket.

3. The safe retractable type syringe as claimed in claim 1, wherein said needle set is a standard retractable type needle set with a changeable needle and comprises said needle, a needle seat and a bottom seat, said needle provided on a front end of said needle seat is adapted to detaching from and connecting to said bottom seat of said needle set.

4. The safe retractable type syringe as claimed in claim 1, wherein said protruding ribs are in shapes of triangles each having a supporting surface, said supporting surface is perpendicular to an inner wall of said front end of said barrel to abut against and support said first flange.

5. The safe retractable type syringe as claimed in claim 1, wherein said at least two protruding ribs are all allocated on a circle, and are equidistantly spaced.
